(19) United States
(12) Patent Application Publication
(10) Pub. No.: US 2021/0137742 A1
(43) Pub. Date: May 13, 2021

(54) HIGH HYGROSCOPIC WOUND DRESSING AND PREPARATION METHOD AND USE THEREOF

(71) Applicant: Foshan United Medical Technologies, Ltd., Foshan (CN)

(72) Inventors: Xiaodong WANG, Foshan (CN); Xiaohui MO, Foshan (CN)

(21) Appl. No.: 17/149,784

(22) Filed: Jan. 15, 2021

Related U.S. Application Data
(63) Continuation-in-part of application No. 14/438,324, filed on Apr. 24, 2015.

(30) Foreign Application Priority Data
Oct. 29, 2012 (CN) 201210421630.7

(51) Int. Cl. A61F 13/00; A61L 15/26; A61L 15/28; A61L 15/60; A61L 15/32; A61L 15/44

(52) U.S. Cl. CPC A61F 13/00042; A61F 13/00012; A61F 13/00987; A61F 13/00017; A61L 15/26; A61L 2300/404; A61L 15/60; A61L 15/32; A61L 15/44; A61F 2013/00238; A61L 15/28

(57) ABSTRACT

The present invention discloses a wound dressing and the method of manufacturing. The wound dressing is a knitted fabric including of a fluffy layer of gel-forming fibers and a backing layer of non-gel-forming fibers. The backing layer includes a plain fabric of the non-gel-forming fibers. The gel-forming fibers are knitted onto the backing layer, a middle part of the gel-forming fibers are held in the backing layer, and two ends of the gel-forming fibers project against the backing layer. The length of the middle part of the gel-forming fibers is no more than the length of one loop of the non-gel-forming fibers, and the length of each of the two ends of the gel-forming fibers is between 1-100 mm.

### CROSS-REFERENCE TO RELATED APPLICATIONS

This application is a continuation-in-part of U.S. patent application Ser. No. 14/438,324, filed on Apr. 24, 2015, which is now pending and is the US national stage of International Patent Application No. PCT/CN2013/082801 with an international filing date of Sep. 2, 2013, which is now abandoned as to the United States and is based on Chinese Patent Application No. 201210421630.7 filed Oct. 29, 2012. The contents of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 14781 Memorial Drive, Suite 1319, Houston, Tex. 77079.

### FIELD OF THE INVENTION

This invention discloses an absorbent wound dressing and its method of manufacturing.

### BACKGROUND OF THE INVENTION

Functionalized wound dressings are preferred in the management of chronic wounds, particularly the wound dressings of gel-forming fibers, such as alginate, chitosan and CMC wound dressings. These dressings have very good absorption and fluid retention properties, and provide moist environment for the wounds. However, all these dressings have a common weakness, i.e., the strength of the dressing is very low, particularly the wet strength. The wet strength is evaluated by soaking the dressing in wound fluid or saline solutions, and then measuring the strength. The low wet strength causes many problems during the dressing changes, such as the dressing being difficult to remove as one piece which may contribute to infections of the surrounding healthy skin.

Normally, chronic wounds produce wound exudates at some stage of healing. The volume of the wound exudate could be as high as 50 ml per 24 hrs. Therefore, there is a need for an absorbent fibrous wound dressing to remove these exudates. At the same time, the dressing should have a good wet strength so that the dressing can be removed intact when it is saturated with wound exudates. By way of illustration with reference to an alginate dressing, typically an alginate dressing can absorb wound fluid up to 20 times its own weight. Commonly the largest alginate dressing is 10×20 cm with a weight of 2-3 grams. After maximal absorption, the dressing would weigh as much as 60 g. This requires the dressing's wet strength to be 60 g (˜0.6N) or higher, so that when the dressing is lifted during dressing change, the dressing will not be torn by its own weight.

US 2009/0287130 uses some stitch bonding to re-enforce the CMC wound dressing (Aquacel). When the dressing absorbs wound fluid, the stitch bonding threads act as the re-enforcement to “hold” the dressing together, making a one-piece removal possible. However, the stitch bonding threads which are made of non-gel-forming fibers) cause the dressing to stick to the wound.

### SUMMARY OF THE INVENTION

The present invention provides an absorbent wound dressing. The wound dressing is applied on wounds for absorbing exudate and is left in place for several days to promote wound healing. The wound dressing comprises a fluffy layer of gel-forming fibers and a backing layer of non-gel-forming fibers. The backing layer comprises a plain fabric of the non-gel-forming fibers. The gel-forming fibers are knitted onto the backing layer, a middle part of the gel-forming fibers forms loops, and are tied in the backing layer, and two ends of the gel-forming fibers are free fibers outside the backing layer. The plain fabric of the non-gel-forming fibers in this invention is obtained through the known plain knitting technology. The plain fabric comprises rows of loops, and the rows of the loops are intermeshed with each other. The free fibers in this invention refer to the two ends of the gel-forming fibers that are not held inside of the backing layer, but project against the backing layer.

The length of the middle part of the gel-forming fibers held in the backing layer is no more than the length of one loop of the non-gel-forming fibers, and the length of each of the two ends of the gel-forming fibers is between 1-100 mm, preferably 5-50 mm.

In one embodiment of the invention, the non-gel-forming fibers are polyester filaments or yarns, nylon filaments or yarns, polyvinyl alcohol (PVA) filaments or yarns, viscose filaments or yarns, Lyocell filaments or yarns, non-gel-forming chitosan filaments or yarns, polyurethane filaments or yarns, polyethylene filaments or yarns, polypropylene filaments or yarns, silk yarns, cotton yarns, or a mixture thereof. The gel-forming fibers are alginate fibers, chemical modified cellulose fibers, chemically modified chitosan fiber, or a mixture thereof. The filament in this invention refers to a fine thread of fibers, and the yarn in this invention refers to a twisted strand of fibers.

In one embodiment of the invention, the absorbency of the dressing of the present invention is at least 6 g/100 cm² or higher according to the test method provided in EN 13726-1:2002/AC:2003. When used in the management of chronic wounds, only the fluffy gel-forming fibers are in contact with the wound bed, and absorb the wound fluid, and the backing layer (made from non-gel-forming fibers) is not significantly weakened by the absorption of the fluid. Accordingly, the dressing is still strong and remains intact at dressing change.

Furthermore, when the dressing is cut in CD (cross machine) direction, the dressing of the present invention curls into a fabric roll with the backing layer inside and the fluffy layer outside. This is ideal for the management of cavity wounds, so that the fluffy layer at the outside of the fabric roll absorbs wound exudates whilst the backing layer at the inside of the fabric roll is kept away from the wound bed and provides strength to allow an intact removal of the dressing.

In one embodiment of the invention, the linear density of the gel-forming fiber is 1-10 dtex, preferably 2-5 dtex.

In one embodiment of the invention, the length of the gel-forming fiber is 10-120 mm, preferably 10-75 mm.

In one embodiment of the invention, the linear density of the non-gel-forming yarn or filament is 50-500 dtex, preferably 50-200 dtex.

The gel-forming fibers in the present invention refer to fibers that form gels when absorbing water or saline or Solution A (contains 8.298 g of sodium chloride and 0.368 g of calcium chloride dihydrate as defined in British Pharmacopoeia 1995). The non-gel-forming fibers in the present invention refer to fibers that do not form gels when absorbing water or saline or Solution A. Solution A is used in the measurement of the dressing's absorbency and wet strength, this is because the Solution A is designed to mimic the sodium and calcium content of the wound fluid. The gel-forming fiber has a very high absorption capacity; typically it absorbs as much as 6 g of the fluid or more per gram of the gel-forming fiber. This kind of fiber also expands laterally enormously, sometimes; the fiber diameter expands into several times or several tens of times the original value due to the absorption of water into the fiber structure (instead of being held between the fibers). Some gel-forming fibers become amorphous, making them more absorbent and weak while wet.

In one embodiment of the invention, the alginate fibers are calcium or calcium/sodium alginate fibers which have an absorbency of 10 g/g or above. In addition to the absorption and gelling properties, the alginate fiber also donates calcium ions which help wound healing and the control of bleeding.

The chemically modified cellulose fibers are carboxymethyl cellulose or sulfonated cellulose fibers, and the sulfonated cellulose fibers are water insoluble. These fibers are originally cellulose fibers such as viscose or lyocell fibers, but have been additionally chemically modified. The modification has added a water absorbing unit to the molecular structure of the fiber which causes the fiber very absorbent and easy to gel. The degree of substitution (DS) of these fibers is typically controlled to between 0.05-0.4.

The chemically modified chitosan fibers are carboxymethyl chitosan fibers or acylated chitosan fibers. Similar to the modification to the cellulose fibers, the chemical structure of the chitosan fiber has also been modified, which has made the fiber very absorbent and easy to gel. The degree of substitution (DS) of these fibers is typically controlled to between 0.1-0.4.

In one embodiment of the invention, part or all gel-forming fibers and/or part or all non-gel-forming fibers contain antimicrobial agent. The typical antimicrobial agent for the gel-forming fibers is silver, iodine, honey or PHMB. The typical antimicrobial agent for the non-gel-forming fibers is silver sodium hydrogen zirconium phosphate, such as Alphasan, which is added to and mixed with the polymer before extrusion therefore making antimicrobial fibers or filaments. Another method is to add silver particles such as silver compound or nano silver metal particles into the polymer solution so that silver particles exist inside the fiber/filament structures and on the surface of the fiber/filament. The wound dressing made from antimicrobial gel-forming fibers or antimicrobial non-gel-forming fibers/filaments is used to manage the infected wounds or wounds which are at risks of infection. Typically such a wound dressing can be applied on wounds for up to 7 days without having to change the dressing. This reduces the cost and pain for the patient.

In one embodiment of the invention, one or both ends of the gel-forming fiber are fixed onto the backing layer through the loops of the backing fabric. The density of the backing fabric is changed by the gauges (thickness) of the needles and by thickness of the filament/yarn. Typically the linear density of the filament/yarn of the backing fabric is around 167 dtex. It is single yarn/filament or double. The density of the backing fabric is typically around 20/inch.

In one embodiment of the invention, the fluffy layer of the dressing comprises of two or more gel-forming fibers.

The wound dressing of the present invention is knitted through a circular knitting machine. The filament/yarn for the backing layer is fed into the machine through a guiding device. The gel-forming fiber for the fluffy layer is made into sliver first then into a carding/feeding device. The typical circular knitting machine is WHCW-T/S18C-27A-1176 which has two basic designs, one is to apply the blow from the front and the other is from the back. The front blow makes one end of the gel-forming fiber gripped by the backing layer; the back blow makes both ends of the gel-forming fiber into the loops of the backing layer. The length of the free fibers of the fabric made by the front blow method is slightly longer than that by the back blow method. The former is 20 mm or longer, the latter is normally 3-10 mm. The average length of the free fibers can also be controlled by cutting to be 1-5 mm.

It is well known that the wet/dry strengths of nonwoven fabrics and their wound dressings are stronger in the cross machine (CD) direction than in the machine direction (MD). Generally, during the dressing change, the dressing will break in the weakest direction.

By combining the gel-forming and non-gel-forming fibers through the knitting technology, the wound dressing of the present invention has very good absorption and fluid retention properties due to the employment of gel-forming fibers in the fluffy layer. Also because the backing layer is made from non-gel-forming fibers, yarns or filaments, the dressing's wet strength is not significantly weakened after the absorption of moisture.

The backing layer of the present invention is knitted from non-gel-forming yarns or filaments. The knitted structure normally has a very good elasticity and strength. The strength of the non-gel-forming yarns or filament is normally unaffected by the absorption of moisture. Therefore, the strength of the backing layer is not reduced significantly when wet, making it possible for an intact removal of the dressing.

Additionally, the present invention discloses a method for manufacturing a wound dressing with high absorption capacity and wet strength. The method includes:

1) Feeding the non-gel-forming yarn or filament into the circular knitting machine to form the backing fabric.

2) Feeding the gel-forming fiber sliver into the feed device of the circular knitting machine.

3) Applying the front blow or back blow to make one end or both ends of the gel-forming fiber held by the loops of the backing layer.

4) Processing the fabric further through cutting and calendaring.

5) Converting the fabric into a dressing through cutting to size, packing into pouches and sterilisation (gamma irradiation or ethylene oxide (EtO) or autoclave).

In step 2 of the above process, the number of slivers fed into the circular knitting machine is varied to control the weight of the fluffy layer. For example, for a circular knitting machine that takes maximum 18 slivers, the actual number of sliver is 6, or 9, or 12, or 18. The linear density of the sliver is between 4-18 g/m, preferably 6-12 g/m.

In step 1 of the above process, the distance between the needles (cross machine) is 6-18 needles/inch. The density of the backing layer is normally 12-42/inch.

The length of the free fiber of the fluffy layer depends on the method of the blow. The front blow makes the free fiber longer, the back blow makes the free fiber shorter. Typically the length of the free fiber is between 5-10 mm. If a cutting step is employed, the length of the free fiber is further reduced to 1-2 mm.

The wound dressing of the present invention combines the gel-forming fibers and the non-gel-forming fibers in a knitted fabric in such a way that the non-gel-forming fibers form loop structure of the knitted fabric (i.e., the backing layer) and only a middle part of the gel-forming fibers is held by the loop structure. The two ends of the gel-forming fibers are not held by the loop structure and becomes the fluffy layer. In this way, the middle part of the gel-forming fibers are tied in the backing layer and become a part of the knitted fabric. The gel-forming fibers provide the absorption capacity of the dressing. The non-gelling fibers of the backing layer provide the strength of the fabric which does not change significantly when the dressing absorbs wound exudates or water.

The wound dressing from the present invention can be used in the management of chronic wounds, such as venous stasis ulcers, pressure ulcers, diabetic foot ulcers and other hard-to-heal wounds. Also the wound dressing of the present invention can be used in the management of cavity wounds.

### BRIEF DESCRIPTION OF THE DRAWINGS

FIG. 1 is an illustration of the backing layer in accordance with one embodiment of the disclosure;

FIG. 2 is an illustration of an absorbent wound dressing in accordance with one embodiment of the disclosure;

FIG. 3 is an illustration of an absorbent wound dressing in accordance with another embodiment of the disclosure.

### EXAMPLE

To further illustrate, embodiments detailing an absorbent wound dressing are described below. It should be noted that the following embodiments are intended to describe and not to limit the disclosure.

As shown in FIG. 1, the disclosure provides an absorbent wound dressing, comprising a fluffy layer of gel-forming fibers 1 and a backing layer of non-gel-forming fibers 2. The backing layer comprises a plain fabric of the non-gel-forming fibers. The plain fabric of the non-gel-forming fibers has a pattern of FIG. 1, and is obtained through the known plain knitting technology. The plain fabric comprises rows of loops of the non-gel-forming fibers 2, and each row of the loops is intermeshed with a preceding row of loops. One loop refers to the non-gel-forming fibers 2 from A to B as shown in FIG. 2. The length of one loop of the non-gel-forming fibers is between 1 and 40 mm, preferably between 2 and 20 mm. The gel-forming fibers 1 are knitted onto the backing layer, a middle part of the gel-forming fibers 1 forms loops, and are held in the backing layer, and the two ends of the gel-forming fibers 1 are not held inside, but project against the backing layer. The length of the middle part of the gel-forming fibers 1 that is held by the back layer is no more than the length of one loop of the non-gel-forming fibers 2, and the length of each of the two ends of the gel-forming fibers is between 1-100 mm, preferably 5-50 mm. Preferably, the length of the middle part of the gel-forming fibers is between 2 and 20 mm. The non-gel-forming fibers 2 are polyester filaments or yarns, nylon filaments or yarns, polyvinyl alcohol (PVA) filaments or yarns, viscose filaments or yarns, Lyocell filaments or yarns, non-gel-forming chitosan filaments or yarns, polyurethane filaments or yarns, polyethylene filaments or yarns, polypropylene filaments or yarns, silk yarns, cotton yarns, or a mixture thereof. The gel-forming fibers 1 are alginate fibers, chemical modified cellulose fibers, chemically modified chitosan fiber, or a mixture thereof.

### Example 1

Feed 2×183 dtex Lyocell filaments into the WHCW-S18C-24B-1056 circular knitting machine. The needle gauge is 14 needles/inch, the backing layer density is 18/inch. The backing layer has a structure as shown in FIG. 1.

Feed the calcium alginate slivers (linear density 6.7 g/m) into the above knitting machine. The fiber linear density is 2.65 dtex and the number of slivers is 18.

The blow method: front.

The fabric obtained has a structure of FIG. 2, and has the weight of 410 g/m².

The length of free fiber of the fluffy layer is 40 mm. The absorbency measured by EN 13726-1:2002/AC:2003 is 45 g/100 cm².

The fabric dry strength in MD is 150 N/cm, elongation is 90%. The fabric wet strength is 145 N/cm, elongation is 93%. The fabric dry strength in CD is 41 N/cm, elongation is 266%. The fabric wet strength is 40 N/cm, elongation is 240%.

### Example 2

Feed 2×167 dtex polyester filaments into the WHCW-S18C-27A-1176 circular knitting machine. The needle gauge is 14 needles/inch, the backing layer density is 20/inch. The backing layer has a structure as shown in FIG. 1.

Feed the calcium/sodium alginate slivers (linear density 6.7 g/m) into the above knitting machine. The fiber linear density is 2.65 dtex and the number of slivers is 9.

The blow method: back.

The fabric obtained has a structure of FIG. 3, and has the weight of 320 g/m².

The length of free fiber of the fluffy layer is 12 mm. The absorbency measured by EN 13726-1:2002/AC:2003 is 32 g/100 cm².

The fabric dry strength in MD is 109 N/cm, elongation is 128%. The fabric wet strength is 100 N/cm, elongation is 115%. The fabric dry strength in CD is 44 N/cm, elongation is 192%. The fabric wet strength is 41 N/cm, elongation is 168%.

### Example 3

Feed 2×167 dtex polyester filaments into the WHCW-S18C-27A-1176 circular knitting machine. The needle gauge is 14 needles/inch, the backing layer density is 20/inch. The backing layer has a structure as shown in FIG. 1.

Feed the carboxymethyl cellulose fiber sliver (linear density 6 g/m) into the above knitting machine. The fiber linear density is 2.1 dtex and the number of slivers is 9.

The blow method: back.

The fabric obtained has a structure of FIG. 3, and has the weight of 300 g/m².

The length of free fiber of the fluffy layer is 8 mm. The absorbency measured by EN 13726-1:2002/AC:2003 is 33 g/100 cm².

The fabric dry strength in MD is 105 N/cm, elongation is 120%. The fabric wet strength is 95 N/cm, elongation is 105%. The fabric dry strength in CD is 42 N/cm, elongation is 182%. The fabric wet strength is 39 N/cm, elongation is 165%.

### Example 4

Feed 2×167 dtex polyester filaments into the WHCW-S18C-27A-1176 circular knitting machine. The needle gauge is 14 needles/inch, the backing layer density is 20/inch. The backing layer has a structure as shown in FIG. 1.

Feed acylated chitosan slivers (linear density 6 g/m) into the above knitting machine. The fiber linear density is 2.2 dtex and the number of slivers is 9.

The blow method: back.

The fabric obtained has a structure of FIG. 3, and has the weight of 290 g/m².

The length of free fiber of the fluffy layer is 5 mm. The absorbency measured by EN 13726-1:2002/AC:2003 is 32 g/100 cm².

The fabric dry strength in MD is 101 N/cm, elongation is 128%. The fabric wet strength is 92 N/cm, elongation is 112%. The fabric dry strength in CD is 39 N/cm, elongation is 180%. The fabric wet strength is 40 N/cm, elongation is 176%.

### Example 5

Feed 2×167 dtex polyester filaments into the WHCW-S18C-27A-1176 circular knitting machine. The needle gauge is 14 needles/inch, the backing layer density is 20/inch. The backing layer has a structure as shown in FIG. 1.

Feed calcium alginate slivers (linear density 6.7 g/m) into the above knitting machine. The fiber linear density is 2.65 dtex and the number of slivers is 9.

The blow method: back.

The fabric obtained has a structure of FIG. 3, and has the weight of 300 g/m².

The length of free fiber of the fluffy layer is 8 mm. The absorbency measured by EN 13726-1:2002/AC:2003 is 30 g/100 cm².

The fabric dry strength in MD is 106 N/cm, elongation is 116%. The fabric wet strength is 197 N/cm, elongation is 103%. The fabric dry strength in CD is 41 N/cm, elongation is 180%. The fabric wet strength is 37 N/cm, elongation is 159%.

### Example 6

Feed 2×167 dtex polyester filaments into the WHCW-S18C-27A-1176 circular knitting machine. The needle gauge is 14 needles/inch, the backing layer density is 20/inch. The backing layer has a structure as shown in FIG. 1.

Feed silver alginate slivers (linear density 6.7 g/m) into the above knitting machine. The fiber linear density is 2.75 dtex and the number of slivers is 9.

The blow method: back.

The fabric obtained has the weight of 320 g/m².

The length of free fiber of the fluffy layer is 12 mm. The absorbency measured by EN 13726-1:2002/AC:2003 is 31 g/100 cm².

The fabric dry strength in MD is 110 N/cm, elongation is 120%. The fabric wet strength is 105 N/cm, elongation is 113%. The fabric dry strength in CD is 39 N/cm, elongation is 190%. The fabric wet strength is 42 N/cm, elongation is 170%.

### Example 7

Feed 2×167 dtex polyester filaments into the WHCW-S18C-27A-1176 circular knitting machine. The needle gauge is 14 needles/inch, the backing layer density is 20/inch. The backing layer has a structure as shown in FIG. 1.

Feed PHMB alginate slivers (linear density 6.7 g/m) into the above knitting machine. The fiber linear density is 2.75 dtex and the number of slivers is 9.

The blow method: back.

The fabric obtained has a structure of FIG. 3, and has the weight of 320 g/m².

The length of free fiber of the fluffy layer is 12 mm. The absorbency measured by EN 13726-1:2002/AC:2003 is 31 g/100 cm².

The fabric dry strength in MD is 101 N/cm, elongation is 124%. The fabric wet strength is 98 N/cm, elongation is 114%. The fabric dry strength in CD is 45 N/cm, elongation is 188%. The fabric wet strength is 40 N/cm, elongation is 166%.

### Example 8

Feed 2×167 dtex polyester filaments into the WHCW-S18C-27A-1176 circular knitting machine. The needle gauge is 14 needles/inch, the backing layer density is 20/inch. The backing layer has a structure as shown in FIG. 1.

Feed silver carboxymethyl cellulose slivers (linear density 6 g/m) into the above knitting machine. The fiber linear density is 2.2 dtex and the number of slivers is 9.

The blow method: back.

The fabric obtained has a structure of FIG. 3, and has the weight of 300 g/m².

The length of free fiber of the fluffy layer is 8 mm. The absorbency measured by EN 13726-1:2002/AC:2003 is 32 g/100 cm².

The fabric dry strength in MD is 103 N/cm, elongation is 118%. The fabric wet strength is 96 N/cm, elongation is 104%. The fabric dry strength in CD is 38 N/cm, elongation is 176%. The fabric wet strength is 35 N/cm, elongation is 143%.

### Example 9

Feed 2×167 dtex polyester filaments into the WHCW-S18C-27A-1176 circular knitting machine. The needle gauge is 14 needles/inch, the backing layer density is 20/inch. The backing layer has a structure as shown in FIG. 1.

Feed silver acylated chitosan slivers (linear density 6 g/m) into the above knitting machine. The fiber linear density is 2.3 dtex and the number of slivers is 9.

The blow method: back.

The fabric obtained has a structure of FIG. 3, and has the weight of 300 g/m².

The length of free fiber of the fluffy layer is 5 mm. The absorbency measured by EN 13726-1:2002/AC:2003 is 31 g/100 cm².

The fabric dry strength in MD is 98 N/cm, elongation is 125%. The fabric wet strength is 90 N/cm, elongation is 117%. The fabric dry strength in CD is 38 N/cm, elongation is 185%. The fabric wet strength is 44 N/cm, elongation is 186%.

What is claimed is:

1. An absorbent wound dressing adapted to be applied on wounds and left in place for several days to promote wound healing, the absorbent wound dressing comprising a backing layer of non-gel-forming fibers, and a layer of gel-forming fibers knitted on the backing layer, wherein:
the gel-forming fibers are alginate fibers, chemically modified cellulose fibers, chitosan fibers, or a mixture thereof;
the non-gel-forming fibers are polyester filaments or yarns, nylon filaments or yarns, PVA filaments or yarns, viscose filaments or yarns, lyocell filaments or yarns, non-gel-forming chitosan filaments or yarns, polyurethane filaments or yarns, polypropylene filaments or yarns, cotton yarns, or a mixture thereof;
the backing layer comprises a plain fabric of the non-gel-forming fibers;
a middle part of the gel-forming fibers forms loops, and are tied in the backing layer, and two ends of the gel-forming fibers project against the backing layer;
a length of the middle part of the gel-forming fibers is no more than a length of one loop of the non-gel-forming fibers, and
a length of each of the two ends of the gel-forming fibers is between 1 and 100 mm.

2. The absorbent wound dressing of claim 1, wherein the length of one loop of the non-gel-forming fibers is between 1 and 40 mm.

3. The absorbent wound dressing of claim 1, wherein the length of one loop of the non-gel-forming fibers is between 2 and 20 mm.

4. The absorbent wound dressing of claim 1, wherein the wound dressing has an absorption capacity of 6 g/100 cm² or more when tested according to EN 13726-1:2002/AC:2003.

5. The absorbent wound dressing of claim 1, wherein the wound dressing, when cut in the cross-machine direction, curls by itself into a fabric roll with the backing layer inside and the layer of the gel-forming fibers outside.

6. The absorbent wound dressing of claim 1, wherein a linear density of the gel-forming fibers is 1-10 dtex.

7. The absorbent wound dressing of claim 1, wherein the length of each of the two ends of the gel-forming fibers is 10-75 mm.

8. The absorbent wound dressing of claim 1, wherein a linear density of the non-gel-forming fibers is 50-500 dtex.

9. The absorbent wound dressing of claim 1, wherein the length of each of the two ends of the gel-forming fibers is between 5 and 50 mm.

10. The absorbent wound dressing of claim 1, wherein the alginate fibers are calcium alginate or calcium/sodium alginate fibers.

11. The absorbent wound dressing of claim 1, wherein the chemically modified cellulose fibers are carboxymethyl cellulose fibers or sulfonated solvent spun cellulose fibers, the sulfonated solvent spun cellulose fibers being water insoluble.

12. The absorbent wound dressing of claim 1, wherein the chitosan fibers are carboxymethyl chitosan fibers or acylated chitosan fibers.

13. The absorbent wound dressing of claim 1, wherein the gel-forming fibers comprise an antimicrobial agent.

14. The absorbent wound dressing of claim 1, wherein the absorbent wound dressing is adapted to be applied on wounds and left in place for up to seven days to promote wound healing.

15. A method of manufacturing the absorbent wound dressing of claim 1, the method comprising:
1) feeding the non-gel-forming fibers into a circular knitting machine to form the backing layer;
2) feeding the gel-forming fiber into the circular knitting machine, and knitting the gel-forming fibers into the backing layer through front blow or back blow to obtain a knitted fabric;
3) cutting the knitted fabric, calendaring; and
4) packing the knitted fabric into pouches, and sterilizing through gamma irradiation, EtO or autoclave.
